(19) United States
(12) Patent Application Publication
(10) Pub. No.: US 2015/0150924 A1
(43) Pub. Date: Jun. 4, 2015

(54) WOUND HEALING COMPOSITE WITH A FUNCTION OF PROMOTING THE GROWTH OF GRANULATION TISSUES

(71) Applicant: Li Tzu Pao Mei Regiman Beauty Public Ltd. Company, Kaohsiung City (TW)

(72) Inventor: Hui-Lien Hsieh, Kaohsiung City (TW)

(21) Appl. No.: 14/569,765

(22) Filed: Dec. 14, 2014

Related U.S. Application Data
(63) Continuation-in-part of application No. 14/094,795, filed on Dec. 3, 2013.

(51) Int. Cl. A61K 36/282; A61K 36/355; A61K 36/16

(52) U.S. Cl. CPC A61K 36/282; A61K 36/16; A61K 36/355

(57) ABSTRACT

A wound healing composite with a function of promoting the growth of granulation tissues includes a substrate extracted from a plant and a base, and the substrate includes a loniceraiaponica extract, a mugwort extract, and ginkgo, and both loniceraiaponica extract and ginkgo have a weight percentage ranging from 0.28% to 3.5%. The wound healing composite can effectively promote the growth of granulation tissues, and loniceraiaponica, mugwort, ginkgo and other ingredients are extracts of natural plants, and thus the wound healing composite has a low drug resistance and does not have the side effect of drug allergy or the doubt leading to skin cancer.

### CROSS-REFERENCE TO RELATED APPLICATIONS

This application is a continuation-in-part of U.S. patent application Ser. No. 14/094,795, filed Dec. 3, 2013, the contents of which are incorporated herein by reference.

### FIELD OF THE INVENTION

The present invention relates to a wound healing composite, and more particularly to the wound healing composite with a function of promoting the growth of granulation tissues.

### BACKGROUND OF THE INVENTION

Present medical research results indicate that a wound healing can be mainly divided into the following four stages:

(1) Blood Clot Formation: Activate blood platelets and start the clotting mechanism in human body;

(2) Infiltration of Fibroblasts, Keratinocytes, Macrophages and other Neutrophils;

(3) Granulation Tissue Formation: Granulation tissue is mainly composed of collagen secreted from fibroblast and capable of replacing the original clot tissue, and the fibroblast is further divided into muscle fibroblast that can help wound contraction; and

(4) Re-epithelialization: Epithelial regeneration of the infiltrated keratinocyte takes place, and the surface of wound is covered by the migration of epithelial cells to reduce pathogenic infection and water loss.

As to most chronic patients such as diabetic patients, the synthesis of collagen is insufficient due to the metabolic disorder of the chronic patients, or the collagen is denatured since the collagen is combined with the too-high blood sugar of the diabetic patients, and thus the wounds of the chronic patients cannot be healed easily. Particularly for elderly long-term bedridden patients, decubitus is very difficult to cure. Most present clinical treatments adopt a cream dressing method to block bacteria at the unhealed wound and maintain moisture, or use antimicrobials or antibiotics to isolate the wound from the contact with the outside.

Although infections can be prevented effectively, these methods do not have any healing effect to the wound. Therefore the chronic patients having problems with the wound healing mechanism generally require a very long time of medicine dressing and waiting for the wound healing. However, the long time of medicine dressing causes a high air permeability of the wound, so that the wound may breed bacteria when the wound is situated at a wet condition for a long time. As a result, the viscous cycle may happen and cause the wound to be healed more uneasily.

Therefore, it is one of the subjects worthy of research to expedite the wound healing speed of chronic patients and prevent their wound from getting more increasingly serious.

### DESCRIPTION OF THE PREFERRED EMBODIMENTS

The technical characteristics and contents of the present invention will become apparent with the detailed description of preferred embodiments as follows:

The present invention discloses a wound healing composite with the function of promoting the growth of granulation tissues, comprising: a substrate extracted from a plant and a base, wherein the substrate includes a loniceraiaponica extract, a mugwort extract and ginkgo, and the base is water.

Wherein, loniceraiaponica has the effects of resisting and curing viruses, fungi, crab itching, swelling, bacteria, external wound infections, cervicitis and various infections and its composition comprises:

1. Volatile Oils;

2. Flavonoids and Chlorogenic Acids including luteolin, inositol, chlorogenic acid and isochlorogenic acid in flower stamens;

3. Trierpenoids such as trierpenoid compounds; and

4. Inorganic Elements including 15 trace elements such as Fe, Mn, Cu, Zn, Ti, Sr, Mo, Ba, Ni, Cr, Pb, V, Co, Li, and Ga.

In general, the loniceraiaponica extract such as loniceraiaponica decoction) used for suppressing pathogenic microorganisms (such as Gram positive negative bacteria) has been proven to have a relatively high suppressing effect on influenza virus, orphan virus and herpes through a tissue cell culture process, particularly on Coxsackie virus or Echo virus. In the meantime, biological experiments show that the loniceraiaponica extract can reduce the rival liver damage in mice.

Artemisiae argy folium has the effects of curing cold, releasing pain, stopping bleeding, and curing skin itching such as eczema itching.

According to Compendium of Materia Medica, ginkgo includes nitric oxide (NO) capable of treating nasal blister scabies, malnutrition worms, night blindness, and blood platelet aggregation as well as expanding blood vessels to improve the blood circulation of skin wounds and has a very good effect on improving blood flow and microvascular permeability as well as blood vessel elasticity. In addition, the composition of ginkgo also further includes minerals and vitamins A, B, B2 and B3 capable of promoting peripheral blood circulation, metabolism, and antioxidation.

Loniceraiaponica has a high content of chlorogenic acids, glycosides, flavonoids, and volatile oils, wherein the composition of glycosides includes saponins and iridoid glycosides such as triterpenoid saponins using ivy aglycone as ligand, triterpenoid saponins using oleanolic acid as aglycone, and the composition of flavonoids includes 5-hydroxy--3,4 (or 7)-trimethoxy flavone, luteolin -7-O-a-D-glucoside, luteolin -7-O-b-D-galactose, quercetin -3-O-b-D-glucoside, and hyperin. In the preparation method, a loniceraiaponica powder (0.1 g) is put into methanol or ethanol (2 mL) to form a loniceraiaponica extract solution, and then dipped into the extract solution at room temperature for 12 hours before it is filtered, and then a methanol solution with a concentration of 70% is added into the loniceraiaponica extract solution until the solution is quantified to 30 mL, and then the loniceraiaponica extract solution is shaken for 20 minutes before it is heated and baked dry to form a loniceraiaponica extract.

In the method of preparing the mugwort extract, artemisiae argy folium powder (5 g) is added into ethanol solution (50 mL) with a concentration of 75%, and then heated for an hour for reflux before the solution is filtered, and the filtered solution is heated and evaporated, and pure water (10 mL) is added to the remaining residue to dissolve the residue, and finally the dissolved residue is finally put into a separatory funnel and extracted twice by chloroform (1 mL for each time), and the chloroform solution is combined, heated, and evaporated, and then methanol (1 mL) is added to the remaining residue to dissolve the residue, and the residue is heated and evaporated to obtain a mugwort extract.

Preferably, the composition of a first preferred embodiment includes a loniceraiaponica extract (10% by weight), a mugwort extract (10% by weight), ginkgo (10% by weight), and water (70% by weight).

Preferably, the composition of a second preferred embodiment includes a loniceraiaponica extract (35% by weight), a mugwort extract (10% by weight), ginkgo (35% by weight), and water (20% by weight).

Preferably, the composition of a third preferred embodiment includes a loniceraiaponica extract (10% by weight), a mugwort extract (10% by weight), ginkgo (35% by weight), and water (45% by weight).

Preferably, the composition of a fourth preferred embodiment includes a loniceraiaponica extract (35% by weight), a mugwort extract (10% by weight), ginkgo (10% by weight), and water (45% by weight).

Preferably, the composition of a fifth preferred embodiment includes a loniceraiaponica extract (30% by weight), a mugwort extract (10% by weight, ginkgo (30% by weight), and water (30% by weight).

In this preferred embodiment, candida albicans with a concentration of 1.8×10⁴ (CFU/mL) is used as an experiment group, and distilled water added into the candida albicans with a concentration of 1.8×10⁴ (CFU/mL) is used as a control group, and the solutions of these two groups are sat still at room temperature for a period of time, and then the remaining quantity of candida albicans is observed and measured as listed in the experiment data of Table 1.

TABLE 1

| | Original Added | Remaining Quantity after |
|---|---|---|
| | Quantity (CFU/mL) | 12 Hours (CFU/mL) |
| Control | 1.8 × 10⁴ | 1.8 × 10⁴ |
| Group | | |
| Experiment | 1.8 × 10⁴ | <10 |
| Group | | |
| Sterilization | | >99.9% |
| Rate | | |

Wherein, CFU represents the number of colonies per unit volume.

In this preferred embodiment, staphylococcus aureus with a concentration of 1.7×10⁵ (CFU/mL) is used as an experiment group, and distilled water added to the staphylococcus aureus with a concentration of 1.7×10⁵ (CFU/mL) is used as a control group, and the solutions of these two groups are sat still at room temperature for a period of time, and then the remaining quantity of staphylococcus aureus is observed and measured as listed in the experiment data of Table 2.

TABLE 2

| | Original Added | Remaining Quantity |
|---|---|---|
| | Quantity (CFU/mL) | after 1 Hour (CFU/mL) |
| Control | 1.7 × 10⁵ | 1.7 × 10⁵ |
| Group | | |
| Experiment | 1.7 × 10⁵ | <10 |
| Group | | |
| Sterilization | | >99.9% |
| Rate | | |

In this preferred embodiment, bacillus dander with a concentration of 1.8×10⁴ (CFU/mL) is used as an experiment group, and distilled water added into the bacillus dander with a concentration of 1.8×10⁴ (CFU/mL) is used as a control group, and the solutions of these two groups are sat still at room temperature for a period of time, and then the remaining quantity of the bacillus dander is observed and measured as listed the experiment data of Table 3.

TABLE 3

| | Original Added | Remaining Quantity after 12 |
|---|---|---|
| | Quantity (CFU/mL) | Hours (CFU/mL) |
| Control | 1.8 × 10⁴ | 1.8 × 10⁴ |
| Group | | |
| Experiment | 1.8 × 10⁴ | 0 |
| Group | | |
| Sterilization | | 100% |
| Rate | | |

In this preferred embodiment, epidermophyton floccosum with of a concentration of 3.2×10⁴ (CFU/mL) is used as an experiment group, and distilled water added to the epidermophyton floccosum with of a concentration of 3.2×10⁴ (CFU/mL) is used a control group, and the solutions of these two groups are sat still at room temperature for a period of time, and then the remaining quantity of epidermophyton floccosum is observed and measured as listed in the experiment data of Table 4.

TABLE 4

| | Original Added | Remaining Quantity after 12 |
|---|---|---|
| | Quantity (CFU/mL) | Hours (CFU/mL) |
| Control | 3.2 × 10⁴ | 3.2 × 10⁴ |
| Group | | |
| Experiment | 3.2 × 10⁴ | 0 |
| Group | | |
| Sterilization | | 100% |
| Rate | | |

From Tables 1 and 2, both candida albicans and staphylococcus aureus of the preferred embodiments achieve the sterilization rate over 99.9% within a short time. In general, candida albicans and staphylococcus aureus are main pathogens causing skin allergies such as candida erosive disease, candidiasis mill rash, and atopic dermatitis. In other words, these preferred embodiments have good effects on the symptoms caused by candida albicans and staphylococcus aureus. From Tables 3 and 4, these preferred embodiments have a 100% sterilization rate on bacillus dander and epidermophyton floccosum within 12 hours. In general, bacillus dander and epidermophyton floccosum are pathogens causing tinea and wound infection. In other words, these preferred embodiments have very good effects on the symptoms caused by bacillus dander and epidermophyton floccosum.

This preferred embodiment can promote the growth of granulation tissues, and loniceraiaponica, artemisiae argy folium, and ginkgo are extracts of natural plants. Compared with the composition of the conventional western medicine, the invention has a low drug resistance and does not have the side effect of drug allergy or the doubt leading to skin cancer, and thus the invention can assist the growth of granulation tissues to promote the protection of skin against environmental pollutions and maintain the elasticity of the skin. In summation, the present invention can achieve the aforementioned objectives.

What is claimed is:

1. A wound healing composite with a function of promoting the growth of granulation tissues, comprising: a substrate extracted from a plant and a base, wherein the substrate includes a loniceraiaponica extract, a mugwort extract, and ginkgo, and both loniceraiaponica extract and ginkgo have a weight percentage falling within a range from 0.28 to 3.5.

2. The wound healing composite with a function of promoting the growth of granulation tissues according to claim 1, wherein the loniceraiaponica extract and ginkgo have a weight percentage equal to 1.

3. The wound healing composite with a function of promoting the growth of granulation tissues according to claim 2, wherein the loniceraiaponica extract and ginkgo have a weight percentage equal to 30%.

4. The wound healing composite with a function of promoting the growth of granulation tissues according to claim 1, wherein the loniceraiaponica extract has a weight percentage falling within a range from 10% to 35%.

5. The wound healing composite with a function of promoting the growth of granulation tissues according to claim 1, wherein, the ginkgo has a weight percentage falling within a range from 10% to 35%.

6. The wound healing composite with a function of promoting the growth of granulation tissues according to claim 1, wherein the mugwort extract has a weight percentage equal to 10%.

7. The wound healing composite with a function of promoting the growth of granulation tissues according to claim 1, wherein the base is water.

8. The wound healing composite with a function of promoting the growth of granulation tissues according to claim 7, wherein the base has a weight percentage falling within a range from 20% to 70%.
